(19) United States
(12) Patent Application Publication
(10) Pub. No.: US 2025/0352556 A1
(43) Pub. Date: Nov. 20, 2025

(54) BUDESONIDE TWO LAYERS CAPSULE FORMULATION

(71) Applicant: Synthon B.V., Nijmegen (NL)

(72) Inventors: Luis NOGUEIRAS NIETO, Sant Boi de Llobregat (ES); Lisardo ALVAREZ FERNANDEZ, Sant Boi de Llobregat (ES); Rohit KUMAR, Sant Boi de Llobregat (ES)

(21) Appl. No.: 18/993,276

(22) PCT Filed: Jul. 11, 2023
(86) PCT No.: PCT/EP2023/069160

§ 371 Date: Jan. 10, 2025

(30) Foreign Application Priority Data
Jul. 15, 2022 (EP) 22185285.8

(51) Int. Cl. A61K 31/58; A61K 9/20; A61K 9/50

(52) U.S. Cl. CPC A61K 31/58; A61K 9/2081; A61K 9/5047; A61K 9/5078; A61K 9/5089

(57) ABSTRACT

The present invention relates to a Budesonide formulation with one or more layers comprising organic acid and Budesonide in the same layer.

### BACKGROUND OF THE PRESENT INVENTION

Budesonide, a synthetic corticosteroid, is designated chemically as 16a, 17a-[(IRS)Butylidenebis(oxy)]-11β, 21-dihydroxypregna-1,4-diene-3,20-dione,

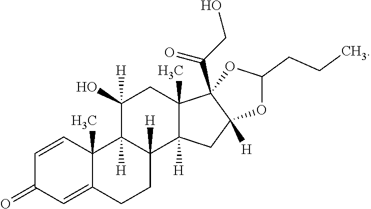

It is marketed as TARPEYOR delayed release capsules 4 mg. TARPEYO® is the first approved treatment to reduce proteinuria in adults with primary immunoglobulin A nephropathy (IgAN) at risk of rapid disease progression. The approved marketed formulation has been disclosed in WO2009138716 and consists of hypromellose capsules coated with methacrylic acid and methacrylate copolymer as delayed, pH-dependent polymer, talc as anticaking agent and dibutyl sebacate as plasticizer; the capsules carry coated, extended-release pellets consisting of an inert sugar pellets (sucrose and starch) as carrier, a first coating layer made of Budesonide, hypromellose and polyethylene glycol, a second coating layer made of citric acid, hypromellose and polyethylene glycol and a third alkaline coating layer, made of ethyl cellulose, médium chain triglycerides, oleic acid, hypromellose, and polyethylene glycol

The coating of the capsules with methacrylate copolymer provides the delayed release feature to the drug product.

The role of the citric acid layer is to prevent the degradation of the drug substance due to the alkaline nature of the third layer, made of ethyl cellulose, medium chain triglycerides, oleic acid, hypromellose and polyethylene glycol.

The addition of an intermediate layer of citric acid and hypromellose and polyethylene glycol presents several disadvantages:

Sealing of drug substance layer: in order to prevent the degradation of the drug substance, it should be guaranteed the complete sealing of the drug substance layer, avoiding direct contact with the third layer. In order to mitigate this risk the weight increase of the citric acid layer might be increased, making the coating step longer in time (costly);

An additional coating layer will increase the cost of goods of the manufacturing, and the cost of the product.

There is still need of finding an additional oral formulation of Budesonide which overcomes the problems of the prior art and is bioequivalent to the commercial Budesonide capsules TARPEYO®.

### DETAILED DESCRIPTION OF THE PRESENT INVENTION

The present invention relates to a Budesonide delayed release formulation with enteric capsules that is bioequivalent to the commercial TARPEYO®.

A first aspect of the invention relates to a Budesonide delayed release capsule formulation comprising a sugar pellet coated with one or more layers comprising Budesonide, an organic acid and ethyl cellulose with alkaline residues wherein the acid is in the same layer than Budesonide.

The formulation of TARPEYO® has to have a very specific release profile, which is a combination of a delayed release to prevent the release of the pellets in the stomach followed from a sustained release that allows the release of the drug over a period of time as it passes through the intestine. This specific profile allows main release of the drug in the ileum which is necessary to treat primary immunoglobulin A nephropathy (IgAN).

The formulation of the prior art consists of two components: a sustained release component and a delayed release component. The sustained release component comprises a first layer with Budesonide, a second layer with acid and a third alkaline layer, with ethyl cellulose. The delayed release component is a capsule that allows the release of the sustained release component from the capsule in the intestine.

The formulation of the prior art, needs to have a physical barrier which is a layer of acid completely sealing the drug substance layer to avoid contact with the third alkaline layer containing ethyl cellulose in order to avoid API stability problems. The addition of an intermediate layer of organic acid presents several disadvantages such as the difficulties to obtain a homogenous layer and the extra costs of adding a step to the process.

The inventors have surprisingly found that in order to avoid the stability problems of Budesonide caused by the alkaline residues contained in the ethyl cellulose layer, the organic acid can be added to the API layer. Placing the organic acid in the drug substance layer will prevent changes on the pH of this layer, even when it is in close contact with the alkaline layer of ethyl cellulose layer without the need of a physical barrier as in the prior art.

Consequently, the intermediate layer will be unnecessary, supressing the need of a costly, time-consuming, challenging manufacturing process (coating step).

By the term “sustained release” is meant that drug is released in the body slowly over an extended period of time.

By the term “delayed release” is meant that the release of the drug is delayed until it passes through the stomach into the small intestine.

By the term “enteric capsule” is meant that the capsule is not either dissolved neither disintegrated in the stomach.

Sugar pellets predominantly comprise sucrose with smaller amounts of other materials added, such as starch and are commercially available.

In the present invention the sustained release part of the capsule formulation comprises a sugar pellet coated with one or more layers.

In one embodiment, the Budesonide delayed release capsule formulation according to the invention comprises:
a) A sustained release part comprising a sugar pellet coated with two layers.
   a. The first layer comprises Budesonide, swelling polymer preferably hypromellose and plasticizer preferably polyethylene glycol and an organic acid;
   b. The second layer of alkaline nature comprises ethyl cellulose medium chain triglycerides, oleic acid, and a swelling polymer preferably hypromellose and plasticizer preferably polyethylene glycol;
b) A delayed release capsules.

This delayed release capsule formulation has the advantage to be more easy to manufacture because only contains two layers.

In another embodiment, the Budesonide delayed release capsule formulation according to the invention comprises:
a) A sustained release part comprising a sugar pellet coated with one layer. The layer comprises Budesonide, swelling polymer preferably hypromellose and plasticizer preferably polyethylene glycol and an organic acid, ethyl cellulose, medium chain triglycerides, oleic acid, alkaline residues;
b) A delayed release capsules.

Swelling polymers are polymers that absorb water and increases its volume. Examples of swelling polymers to be used in the above embodiments are hypromellose, hydroxypropyl cellulose (HPC), hydroxyethyl cellulose, methylcellulose, carboxy methyl cellulose (PMC), polyethylene oxide, xanthan gum and sodium alginate. A preferred swelling polymer is hypromellose.

The swelling polymers preferably are used in an amount in the first layer between 1% to 25%, preferably between 3% to 20%, more preferably between 4% to 15% and even more preferably between 5% to 12% by weight based on the total weight of the coated pellet composition.

Plasticizers are components that increases the flexibility and plasticity of the coating film by promoting the pliability of the polymer.

Examples of plasticizer are polyethylene glycol, triacetin, triethyl citrate, tributyl citrate, dibutyl sebacate, propylene glycol. A preferred plasticizer to be used in the above embodiments is polyethylene glycol.

The plasticizer are used in an amount in the first layer between 0.1% to 2.5%, preferably between 0.3% to 2%, more preferably between 0.4% to 1.5% and even more preferably between 0.5% to 1.2% by weight based on the total weight of the coated pellet composition.

Examples of acids suitable to be used in the above embodiments are citric acid, glutamic acid, lactic acid, tartaric acid, fumaric acid, maleic acid, adipic acid and malic acid a preferred acid is citric acid.

The acid of the present invention can be used in a range between 0.05, to 1%, preferably between 0.05% to 0.75%, more preferably between 0.05% to 0.5%, even more preferably between 0.05% and 0.40%, and more preferably between 0.1% and 0.3% by weight based on the total weight of the coated pellet composition.

The ethyl cellulose component containing alkaline residues to be used in the above embodiments is obtained via spraying an ethyl cellulose derivative composition comprising a alkaline component most preferably Surelease® which comprises ethyl cellulose, ammonium hydroxide, medium chain triglycerides, oleic acid and water. During spraying the aqueous components (containing the alkaline ammonium hydroxide) are evaporated, however some residues of ammonium hydroxide stay in the composition providing the alkaline nature of the layer.

The sustained release part of the capsule of the invention is manufactured by methods known by the skilled person, such as film-coating by bottom spray (e.g.: Würster setting) using fluid bed technology.

Further, the capsule formulation of the present invention can comprise other pharmaceutical acceptable excipients, chosen from, for example, diluents, binders, disintegrants and antioxidants.

Examples of antioxidant to be added are BHT or BHA. The antioxidant may be added in the layer containing the acid.

In a preferred embodiment the sustained release part of the capsule of the present invention is prepared dissolving hypromellose and polyethylenglycol in an aqueous solvent, preferably purified water; to this solution an organic acid is added followed by addition of Budesonide, the resulting suspension is spray dryed on the sugar pellets in a fluid bed. These pellets coated with a first layer are again coated in a fluid bed with a dispersion of Surelease® in a solution of hypromellose/polyethylenglycol resulting in sugar pellets with two layers of coating.

In another embodiment the sustained release part of the capsule of the present invention is prepared dissolving hypromellose and polyethylenglycol in an aqueous solvent, preferably purified water, to this solution an organic acid is added followed by addition of Budesonide and surelease®, the resulting dispersion is sprayed on the pellets in the fluid bed, resulting in sugar pellets with one layer of coating.

This sustain release part manufactured according to the above methods is encapsulated on an enteric release capsules.

The present invention is illustrated by the following Examples.

### Example 1

85.7 grams of Opadry clear is weighted and added into 1008 mL of purified water under stirring until complete dissolution after 45 minutes, obtaining a homogenous solution (1). 1.8 grams of anhydrous citric acid are weighed and added to the previous solution (1) under stirring until complete dissolution, obtaining a homogenous solution (2). 24.5 grams of Budesonide are weighted and added to the previous solution (2) under stirring until its complete dispersion during 15 minutes, obtaining a homogenous suspension (3).

1483 grams of sugar pellets are weighted and placed inside of the fluid bed Glatt 1.1. with Würster settings. The sugar pellets are initially heated up to 48-50° C.; then, the suspension (3) is pumped and sprayed into the fluid bed, coating the surface of the sugar pellets. Once the complete suspension (3) is sprayed, a drying step is followed until stable outlet air humidity is achieved. At the end of this manufacturing step, coated pellets (4) are obtained.

9.5 grams of Opadry clear is weighted and added into 272 grams of purified water under stirring until complete dissolution after 45 minutes, obtaining a homogenous solution (5). 190.8 grams of Surelease® dispersion are weighted and added to the previous Opadry solution (5) under stirring; additional 100 grams of purified water are used to rinse Surelease® container and are added to the previous Opadry® solution (5) under stirring. The dispersion is under stirring during additional 15 minutes, obtaining a homogenous dispersion (6).

The previous coated pellets (4) are heated up to 48-50° C.; then, the dispersion (6) is pumped and sprayed into the fluid bled, coating the surface of the coated pellets. Once the complete dispersion (6) is sprayed, a drying step is followed until stable outlet air humidity is achieved. Then, a curing step is followed, keeping the coated pellets at a constant temperature of 50° C. during 30 minutes. At the end of this manufacturing step, coated pellets (7) with two coating layers are obtained.

275 mg of the resulting coated pellets (7) are then encapsulated inside immediate release capsules based on hydroxypropyl mehtylcellulose, resulting in the final product (9).

### Example 2

207 grams of Opadry clear is weighted and added into 2140 mL of purified water under stirring until complete dissolution after 45 minutes, obtaining a homogenous solution (1). 2.1 grams of anhydrous citric acid are weighed and added to the previous solution (1) under stirring until complete dissolution, obtaining a homogenous solution (2). 28.6 grams of Budesonide are weighted and added to the previous solution (2) under stirring until its complete dispersion during 15 minutes, obtaining a homogenous suspension (3).

1730 grams of sugar pellets are weighted and placed inside of the fluid bed Glatt 1.1. with Würster settings. The sugar pellets are initially heated up to 48-50° C.; then, the suspension (3) is pumped and sprayed into the fluid bed, coating the surface of the sugar pellets. Once the complete suspension (3) is sprayed, a drying step is followed until stable outlet air humidity is achieved. At the end of this manufacturing step, coated pellets (4) are obtained.

11.1 grams of Opadry clear is weighted and added into 334 grams of purified water under stirring until complete dissolution after 45 minutes, obtaining a homogenous solution (5). 222.6 grams of Surelease® dispersion are weighted and added to the previous Opadry solution (5) under stirring; additional 100 grams of purified water are used to rinse Surelease® container and are added to the previous Opadry® solution (5) under stirring. The dispersion is under stirring during additional 15 minutes, obtaining a homogenous dispersion (6).

The previous coated pellets (4) are heated up to 48-50° C.; then, the dispersion (6) is pumped and sprayed into the fluid bled, coating the surface of the coated pellets. Once the complete dispersion (6) is sprayed, a drying step is followed until stable outlet air humidity is achieved. Then, a curing step is followed, keeping the coated pellets at a constant temperature of 50° C. during 30 minutes. At the end of this manufacturing step, coated pellets (7) with two coating layers are obtained.

290 mg of the resulting coated pellets (7) are then encapsulated inside enteric capsules based on hydroxypropyl methylcellulose acetate succinate, resulting in the final product (8).

1. A Budesonide delayed release capsule formulation comprising a sugar pellet coated with one or more layers comprising Budesonide, an organic acid and ethyl cellulose with alkaline residues wherein the acid is in the same layer than Budesonide.

2. The Budesonide delayed release capsule formulation according to claim 1 comprising:
a) a sustained release layer comprising sugar pellet coated with two layers:
(i) the first layer comprises Budesonide, swelling polymer and plasticizer and an organic acid;
(ii) the second layer comprises ethyl cellulose medium chain triglycerides, oleic acid, with alkaline residues, swelling polymer and plasticizer; and
b) a delayed release capsule.

3. The Budesonide delayed release capsule formulation according to claim 2, wherein the organic acid is citric acid.

4. The Budesonide delayed release capsule formulation according to claim 2, wherein the swelling polymer in the first and second layers is hypromellose.

5. The Budesonide delayed release capsule formulation according to claim 4, wherein hypromellose is used in an amount between 5% to 12% by weight based on the total weight of the coated pellet composition.

6. The Budesonide delayed release capsule formulation according to claim 2, wherein the plasticizer in the first and second layers is polyethylene glycol.

7. The Budesonide delayed release capsule formulation according to claim 2, wherein the range of acid is 0.1% to 0.3% by weight based on the total weight of the coated pellet composition.

8. A process for making the capsule according to claim 2, which comprises:
a) dissolving swelling polymer and plasticizer to form a solution;
b) adding to the solution of step a) Budesonide and an organic acid to form a suspension;
c) spray drying the suspension of step b) onto sugar pellets to form coated sugar pellets;
d) coating the coated sugar pellets of step c) with an alkaline dispersion of ethyl cellulose, medium chain triglycerides, oleic acid, swelling polymer and plasticizer preferably to form coated pellets; and
e) encapsulating the coated pellets of step d) into delay release capsules.

9. The process according to claim 8, wherein said swelling polymer in steps a) and d) is hypromellose.

10. The process according to claim 9, wherein said plasticizer in steps a) and d) is polyethylene glycol.
